SEQUENCE LISTING (6 sequences)

SEQ ID NO: 1 - DNA, 4977 bp, Homo sapiens
  5′UTR (1)..(209)
  CDS (210)..(1973)
  3′UTR (1974)..(4977)
  polyA_signal (4953)..(4958)
  polyA_site (4977)..(4977)
caagataccctccaggccctcatcaccttttttcaagtcaagatttcatcccatacatgcatgactcaatcagatttggaaatgtggatatgccctgtcgtccaagcccaatatagcccttcccctccaggttccagttatgcggtcgtcagacatacagctcggaatacaccacggagatcatgaaccccgactacaccaagctgaccatggaccttggcagcactgagatcacggctacagccaccacgtccctgcccagcatcagtacctttgtggagggctactcgagcaactacgaactcaagccttcctgcgtgtaccaaatgcagcggcccttgatcaaagtggaggaggggcgggcgcccagctaccatcaccatcaccaccaccaccaccaccaccaccaccatcaccagcagcagcatcagcagccatccattcctccagcctccagcccggaggacgaggtgctgcccagcacctccatgtacttcaagcagtccccaccgtccacccccaccacgccggccttccccccgcaggcgggggcgttatgggacgaggcactgccctcggcgcccggctgcatcgcacccggcccgctgctggacccgccgatgaaggcggtccccacggtggccggcgcgcgcttcccgctcttccacttcaagccctcgccgccgcatccccccgcgcccagcccggccggcggccaccacctcggctacgacccgacggccgctgccgcgctcagcctgccgctgggagccgcagccgccgcgggcagccaggccgccgcgcttgagggccacccgtacgggctgccgctggccaagagggcggccccgctggccttcccgcctctcggcctcacgccctcccctaccgcgtccagcctgctgggcgagagtcccagcctgccgtcgccgcccagcaggagctcgtcgtctggcgagggcacgtgtgccgtgtgcggggacaacgccgcctgccagcactacggcgtgcgaacctgcgagggctgcaagggctttttcaagagaacagtgcagaaaaatgcaaaatatgtttgcctggcaaataaaaactgcccagtagacaagagacgtcgaaaccgatgtcagtactgtcgatttcagaagtgtctcagtgttggaatggtaaaagaagttgtccgtacagatagtctgaaagggaggagaggtcgtctgccttccaaaccaaagagcccattacaacaggaaccttctcagccctctccaccttctcctccaatctgcatgatgaatgctcttgtccgagctttaacagactcaacacccagagatcttgattattccagatactgtcccactgaccaggctgctgcaggcacagatgctgagcatgtgcaacaattctacaacctcctgacagcctccattgatgtatccagaagctgggcagaaaagattccgggatttactgatctccccaaagaagatcagacattacttattgaatcagcctttttggagctgtttgtcctcagactttccatcaggtcaaacactgctgaagataagtttgtgttctgcaatggacttgtcctgcatcgacttcagtgccttcgtggatttggggagtggctcgactctattaaagacttttccttaaatttgcagagcctgaaccttgatatccaagccttagcctgcctgtcagcactgagcatgatcacagaaagacatgggttaaaagaaccaaagagagtcgaagagctatgcaacaagatcacaagcagtttaaaagaccaccagagtaagggacaggctctggagcccaccgagtccaaggtcctgggtgccctggtagaactgaggaagatctgcaccctgggcctccagcgcatcttctacctgaagctggaagacttggtgtctccaccttccatcattgacaagctcttcctggacaccctacctttctaatcaggagcagtggagcagtgagctgcctcctctcctagcacctgcttgctacgcagcaaagggataggtttggaaacctatcatttcctgtccttccttaagaggaaaagcagctcctgtagaaagcaaagactttcttttttttctggctcttttccttacaacctaaagccagaaaacttgcagagtattgtgttggggttgtgttttatatttaggcattgggggatggggtgggagggggttatagttcatgagggttttctaagaaattgctaacaaagcacttttggacaatgctatcccagcaggaaaaaaaaggataatataactgttttaaaactctttctggggaatccaattatagttgctttgtatttaaaaacaagaacagccaagggttgttcgccagggtaggatgtgtcttaaagattggtcccttgaaaatatgcttcctgtatcaaaggtacgtatgtggtgcaaacaaggcagaaacttccttttaatttccttcttcctttattttaacaaatggtgaaagatggaggattacctacaatcagacatggcaaaacaatatggctgtttgcttcataacagctgcaatttttaaagtgctgtcttactaagtcttgttatactctcttatctatatgccggaaataaaaaggaggcagtcatgttagcaaatgacacgttaatatccctagcagaggctgtgttcaccttccctgtcgatcccttctgaggtatggcccatccaagacttttaggccattcttgatggaaccagatccctgccctgactgtccagctatcctgaaagtggatcagattataaactggattacatgtaactgttttggttgtgttctatcaaccccaccagagttccctaaacttgcttcagttatagtaactgactggtatatcattcagaagcgccataagtcagttgagtattgatcctagataagaacatgcaatcagaggactggtcatacagggtaagcaccagggacaataaggatttttatagatataatttaatttttgttattggttaaggagacaattttggagagcaagcaaatcttctttttaaaaaatagtatgaatgtgaatactagaaaagatttaagaaatagtatgagtgtgagtcataggaaggattagtgggctgcgtttcaacattccgtgttcgtactcccttttgtatgtttctactgttaatgccatattactatgagataatttgttgcatagtgtccttatttgtataaacatttgtatgcacgttatattgtaatagctttgcctgtatttattgcaagaccaccagctcctggaagctgagttacagagtaattaaatggggtgttcacagtgacttggatacaccaattagaaattaaataagcaaatatatatatatatatataaatatagcaggttacatatatatatttataatgtgtctttttattaaccatttgtacaataaatgtcacttcccatgccgttattttatggttcatttgcagtgacttttaaggcagtactgtttagcactttgatattaaaattttgcttatgttttgctaaattcgaataatgtttgaagatttttaggtctaaaagtctttatattatatacgctgtatcaagtcaaaatatctttggccattttgctaagaaacaaactttgaatgtcaaactgatgtcacagtagtttttgttagctttaaatcatttttgctttagtctttttaaaggaaaataacaaaactatgctgtttatattgcattaattatacaatcaaacaaatgccaaatgaattgcctaattgcgcaagataacccagataggaaatcatatgtttttttccaagagtcattctaatatttgattatgttatgtgtgcttttatgaaagattgttatttttatatatcaagatgatagaacctggaatgttaggattttgaaatgttagacttggaaggggcctggtctgtcaactagtccaaccccttaaaattcatagaggacgaaactggggcccaattgaagggtgaagagttactcaaggtcaaacagctggtaacagaatcaagactaagacctaatttacctttccatactctttttttttctcaacttcatctatataaaatcaggcttttaaacataaccactaatatttacctgaagataaccatgagtaaagtatacttttgcattaattttttgagtatatgcaaacataataaatattattaaatatcaaggaaagctaacatttcatacaagatagcttcagaccaaattcaaattgaatttgaataaattagaaatactgtgcatacataaccttcttgtgcaccatgagtatttggaaagttaatccttgtttttgtcgtgtctatagagaacaaacaaataaaaacagagccctagagaaatggcgttactttttatttttacacccatcagatttaaggaaaagactttttagccattataatctagtggttggaaggaatgaagaagcttttttagtaataggtccagatatgagtgctaaaaataaagatgatagcatgttcttctgtcttccatagttattacaactatgagagcctcccaagtcatcttatcaactcaacccccttttttttgtcttaatgttgcacataagtttatacagagtggatgaccacactagcacagaagagaacaacatgtattaaagcaggtgattcctcccttggcgggagagctctctcagtgtgaacatgccttctgtgggcggaaatcaggaagccaccagctgttaatggagagtgccttgcttttatttcagacagcagagttttccaaagtttctctgctcctctaacagcattgctctttagtgtgtgttaacctgtggtttgaaagaaatgctcttgtacattaacaatgtaaatttaaatgattaaattacattttatcaatggca

SEQ ID NO: 2 - protein, 587 aa, Homo sapiens
MDLGSTEITATATTSLPSISTFVEGYSSNYELKPSCVYQMQRPLIKVEEGRAPSYHHHHHHHHHHHHHHQQQHQQPSIPPASSPEDEVLPSTSMYFKQSPPSTPTTPAFPPQAGALWDEALPSAPGCIAPGPLLDPPMKAVPTVAGARFPLFHFKPSPPHPPAPSPAGGHHLGYDPTAAAALSLPLGAAAAAGSQAAALEGHPYGLPLAKRAAPLAFPPLGLTPSPTASSLLGESPSLPSPPSRSSSSGEGTCAVCGDNAACQHYGVRTCEGCKGFFKRTVQKNAKYVCLANKNCPVDKRRRNRCQYCRFQKCLSVGMVKEVVRTDSLKGRRGRLPSKPKSPLQQEPSQPSPPSPPICMMNALVRALTDSTPRDLDYSRYCPTDQAAAGTDAEHVQQFYNLLTASIDVSRSWAEKIPGFTDLPKEDQTLLIESAFLELFVLRLSIRSNTAEDKFVFCNGLVLHRLQCLRGFGEWLDSIKDFSLNLQSLNLDIQALACLSALSMITERHGLKEPKRVEELCNKITSSLKDHQSKGQALEPTESKVLGALVELRKICTLGLQRIFYLKLEDLVSPPSIIDKLFLDTLPF

SEQ ID NO: 3 - DNA, 2464 bp, Homo sapiens
  CDS (111)..(1907)
  polyA_signal (2442)..(2447)
  polyA_site (2464)..(2464)
cggaaacttgggggagtgcacagaagaacttcgggagcgcacgcgggaccagggaccaggctgagactcggggcgccagtccgggcaggggcagcgggacgcggccggagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcacgttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccactggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtccaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccttgcacacctggattcagggcccagcactgccaaactggactactccaagttccaggagctggtgctgccccactttgggaaggaagatgctggggatgtacagcagttctacgacctgctctccggttctctggaggtcatccgaaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgcctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttg

SEQ ID NO: 4 - protein, 598 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGPSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVLAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 5 - DNA, 1572 bp, Mus musculus
  CDS (8)..(1537)
aaacaagatgccgtcgggtttccagcagatcggctctgacgatggggaaccccctcggcagcgagtgactggaacactggtcctagctgtattctcagctgtgcttggctcccttcagtttggctataacattggggttatcaatgccccacagaaggtgattgaacagagctacaatgcaacgtggctgggtaggcaaggtcctgggggaccggattccatcccacaaggcaccctcactacgctctgggctctctccgtggccatcttctctgtgggtggcatgatctcttcctttctcattggcatcatttctcaatggttgggaaggaaaagggctatgctggccaacaatgtcttggccgtgttggggggcgccctcatgggcctagccaatgccgcggcctcctatgagatactcattcttggacggttcctcattggcgcctactcagggctaacatcagggctggtgcccatgtatgtgggagaaatcgcccccactcatcttcggggtgccttgggaacactcaaccaactggccatcgtcattggcattctggttgcccaggtgctgggcttggagtctatgctgggcacagctaccctgtggccactgcttctggctctcacagtactccctgctctcctgcagctgattctgctgcccttctgtcctgagagccccagatacctctacatcatccggaacctggaggggcctgcccgaaagagtctaaagcgcctgaccggctgggctgatgtgtctgacgcactagctgagctgaaggatgagaaacggaagttggagagagagcgtccaatgtccttgctccagctcctgggcagccgcacccaccggcagcctctgatcatcgcagtggtgctgcagctgagccaacagctctcaggcatcaatgctgttttctactattcaaccagcatcttcgagtcggctggggtgggacagccagcctacgccaccataggagctggtgtggtcaatacggtcttcacgttggtctcggtgctcttagtagaacgagctggacgacggacactccatctgttgggcctggccggcatgtgtggctgtgccatcttgatgaccgtggctctgctgctgctggaacgggttccagccatgagctatgtctccatcgtggccatatttggctttgtggccttctttgagattggccctggccccattccctggttcattgtggcagagctcttcagccagggcccccgcccagccgccatggctgtcgctggtttctccaactggacctgtaacttcattgtcggcatgggtttccagtatgttgcggatgctatgggtccttacgtcttccttctatttgccgtcctcctgcttggcttcttcatcttcaccttcctaaaagtgcctgaaaccagaggccggacgtttgaccagatctcagctgccttccgacggacaccttcccttttagagcaggaggtgaaacccagtacagaacttgaatacttagggccagatgagaatgactgaggggcaaaacagggtgggagagccaccctctccac

SEQ ID NO: 6 - protein, 509 aa, Mus musculus
MPSGFQQIGSDDGEPPRQRVTGTLVLAVFSAVLGSLQFGYNIGVINAPQKVIEQSYNATWLGRQGPGGPDSIPQGTLTTLWALSVAIFSVGGMISSFLIGIISQWLGRKRAMLANNVLAVLGGALMGLANAAASYEILILGRFLIGAYSGLTSGLVPMYVGEIAPTHLRGALGTLNQLAIVIGILVAQVLGLESMLGTATLWPLLLALTVLPALLQLILLPFCPESPRYLYIIRNLEGPARKSLKRLTGWADVSDALAELKDEKRKLERERPMSLLQLLGSRTHRQPLIIAVVLQLSQQLSGINAVFYYSTSIFESAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTCNFIVGMGFQYVADAMGPYVFLLFAVLLLGFFIFTFLKVPETRGRTFDQISAAFRRTPSLLEQEVKPSTELEYLGPDEND

(19) United States
(12) Patent Application Publication
(10) Pub. No.: US 2006/0234910 A1
(43) Pub. Date: Oct. 19, 2006

(54) Methods for the treatment of insulin resistance and disease states characterized by insulin resistance

(76) Inventors: W. Timothy Garvey, Mountain Brook, AL (US); Yuchang Fu, Mountain Brook, AL (US); Mitchell Lee Martin, Nutley, NJ (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(21) Appl. No.: 11/388,155

(22) Filed: Mar. 23, 2006

Related U.S. Application Data
(60) Provisional application No. 60/664,792, filed on Mar. 24, 2005.

(51) Int. Cl. A61K 38/28; A61K 31/155; A61K 31/175

(52) U.S. Cl. 514/2; 514/3; 514/592; 514/635

(57) ABSTRACT

The MINOR and TR3 genes are disclosed to be insulin responsive genes differentially expressed as a function of insulin resistance and Type 2 Diabetes in humans and a variety of well characterized animal models. The present disclosure shows that MINOR and TR3 have a functional role in insulin resistance. Therefore, MINOR and TR3 are attractive and novel therapeutic targets for combating insulin resistance and/or disease states and conditions characterized by insulin resistance. Correlation of insulin resistance and disease states and conditions characterized by insulin resistance, including Type II diabetes, with MINOR and TR3 gene expression are also disclosed. Methods of diagnosis, treatment and prevention are likewise disclosed for the treatment and/or prevention of insulin resistance and disease states or conditions characterized by insulin resistance.

### CROSS REFERENCE TO RELATED APPLICATION

This patent application claims priority to and the benefit of U.S. Provisional Patent Application No. 60/664,792 filed 24 Mar. 2005.

### FIELD OF THE DISCLOSURE

The present disclosure is directed to methods for the treatment and/or prevention of insulin resistance and disease states and conditions characterized by insulin resistance, and more particularly for the treatment and/or prevention of insulin resistance in skeletal muscles via novel drug targets, biomarkers and gene expression. Such methods may be used to treat a subject suffering from insulin resistance and a variety of disease states and conditions characterized by insulin resistance or to prevent the occurrence insulin resistance or disease states and conditions characterized by insulin resistance in an at risk subject.

### BACKGROUND

Diabetes is a disease caused by defects in insulin secretion and/or defects in a subject's response to the effects of insulin (i.e., insulin resistance), resulting in dysregulation of glucose metabolism. There are two major forms of diabetes: Type 1 diabetes and Type 2 diabetes. Type 1 diabetes is typically an autoimmune disease characterized by the loss of pancreatic β-cell function and an absolute or substantially absolute deficiency of insulin production. In the US, ˜5% of patients with diabetes suffer from Type 1 diabetes. The remainder of the diabetic population (˜95%) suffers from Type 2 diabetes, which is primarily due to defects in one or both of two physiological processes: 1) pancreatic insufficiency, a deficiency in secretion of insulin from the pancreas in response to rising blood glucose levels such as following a meal; and, 2) insulin resistance, the inability of target tissues of insulin action, primarily skeletal muscle, fat and liver, to respond to the hormone. The result of these defects is elevated blood glucose leading to glucose-mediated cellular toxicity and subsequent morbidity (nephropathy, neuropathy, retinopathy, etc.). Insulin resistance is strongly correlated with the development of Type 2 diabetes.

Insulin resistance is also associated with a number of disease states and conditions and is present in approximately 30-40% of non-diabetic individuals. These disease states and conditions include, but are not limited to, pre-diabetes and metabolic syndrome (also referred to as insulin resistance syndrome) (1-3). Pre-diabetes is a state of abnormal glucose tolerance characterized by either impaired glucose tolerance (IGT) or impaired fasting glucose (IFG). Patients with pre-diabetes are insulin resistant and are at high risk for future progression to overt Type 2 diabetes. Metabolic syndrome is an associated cluster of traits that include, but is not limited to, hyperinsulinemia, abnormal glucose tolerance, obesity, redistribution of fat to the abdominal or upper body compartment, hypertension, dysfibrinolysis, and a dyslipidemia characterized by high triglycerides, low HDL-cholesterol, and small dense LDL particles (1-4). Insulin resistance has been linked to each of the traits, suggesting metabolic syndrome and insulin resistance are intimately related to one another. The diagnosis of metabolic syndrome is a powerful risk factor for future development of Type 2 Diabetes, as well as accelerated atherosclerosis resulting in heart attacks, strokes, and peripheral vascular disease.

As skeletal muscle is the predominant target tissue for insulin-mediated glucose uptake (responsible for approximately 80-95% of glucose uptake), and is a critical locus of insulin resistance, defects in glucose uptake in skeletal muscle is a predominate contributor to the clinical manifestations of insulin resistance. The molecular basis for insulin resistance is not fully understood, but appears to involve defects in insulin signal transduction and abnormal cellular trafficking of glucose transporter proteins (5). The effect of insulin on gene expression in human muscle has not been extensively studied, and most prior studies focused on single genes or small numbers of genes with a limited focus (6-8).

Therefore, insulin resistance is a component in the pathogenesis of multiple human disease states and conditions including, but not limited to, metabolic syndrome, pre-diabetes, polycystic ovary syndrome, type 2 diabetes, dyslipidemia, obesity, infertility, inflammatory disorders, cancer, inflammatory diseases, Alzheimer's disease, hypertension, atherosclerosis, cardiovascular disease and peripheral vascular disease. Treatment modalities that combat insulin resistance can be used to effectively treat and prevent not only insulin resistance per se, but also disease states or conditions characterized by insulin resistance. Since in normoglycemic (normal glycemic) individuals skeletal muscle is responsible for approximately 80-95% of the glucose taken up from the blood in response to insulin, insulin resistance in skeletal muscle represents a primary target tissue for anti-diabetic therapies and key source of candidate drug targets.

### SUMMARY OF THE INVENTION

The present disclosure is directed to methods for the treatment and/or prevention of insulin resistance and the disease states and conditions characterized by insulin resistance, such as but not limited to Type II diabetes, and more particularly for the treatment and/or prevention of insulin resistance via novel drug targets, biomarkers and gene expression. The methods disclosed may be used to treat a subject suffering from insulin resistance and/or a variety of disease states and conditions characterized by insulin resistance. The methods disclosed may also be used to prevent the occurrence insulin resistance or disease states and conditions characterized by insulin resistance in an at risk subject.

The disclosure also demonstrates that the MINOR and TR3 genes are components of a general pathway that is involved in the insulin-mediated uptake of glucose from the blood. The present disclosure shows that insulin increases the expression of the MINOR and TR3 genes and further shows that increased expression of the MINOR gene enhances insulin-responsive glucose transport and enhances insulin-mediated recruitment of the GLUT-4 glucose transporters to the plasma membrane. The present disclosure also provides methods of diagnosing a susceptibility to insulin resistance and/or disease states and conditions characterized by insulin resistance in an individual in need of such diagnosis, such as for example Type II diabetes by detecting the activity and/or expression of the MINOR and/or TR3 gene and its concurrent pathway. The present disclosure also provides for methods to treat and/or prevent insulin resistance in a subject in need of such treatment or prevention by activating the MINOR and/or TR3 pathway. The present disclosure provides for methods to treat and/or prevent disease states and conditions characterized by insulin resistance in a subject in need of such treatment or prevention by activating the MINOR and/or TR3 pathway.

### BRIEF DESCRIPTION OF THE FIGURES

FIG. 1 shows MINOR gene expression in human tissues. Northern blot analysis was performed to examine MINOR gene expression in human tissues. Human multiple tissue northern blot was purchased from Clontech (Palo Alto, Calif.) and used as per manufacturer's instructions. Each of the lanes contained mRNA from the specific human tissue and the amount of each RNA blotted on the membrane was normalized with the β-actin cDNA control probe. The probe for detecting the MINOR gene hybridization signal was a 1.1 kb cDNA fragment corresponding to nucleotides 3874 to 4976 (17).

FIGS. 2A-C show MINOR and TR3 gene expression in skeletal muscles of diabetic and insulin resistant rats and mice. The skeletal muscle tissues from diabetic and insulin resistant rats or mice were homogenized and the mRNAs were extracted for cDNA synthesis. Quantitative real-time PCR was used to measure expression of the MINOR and TR3 genes. FIG. 2A shows a comparison of MINOR and TR3 gene expression between streptozotocin-induced diabetic rats (STZ Rat) and Zucker diabetic fatty rats (ZDF Rat).

FIG. 2B shows a comparison of MINOR and TR3 gene expression between ob/ob mice and control mice (ob/ob designates an insulin resistant mouse model in which a defect in the adipocyte-derived hormone leptin is expressed; control mice indicate mice that lack the deficit). FIG. 2C show a comparison of MINOR and TR3 gene expression between db/db mice and control mice (db/db designates an insulin resistant and diabetic mouse model in which a defect in the leptin receptor is expressed; control mice indicate mice that lack the deficit). All results represent the mean i SE from three separate experiments.

FIGS. 3A-3C show the effect of insulin stimulation on MINOR and TR3 gene expression in 3T3-L1 adipocytes. FIGS. 3A and 3B show insulin induces the expression of MINOR and TR3 gene expression in 3T3-L1 adipocytes. Fully differentiated 3T3-L1 adipocytes were treated with 100 nM of insulin for up to 8 hours (treated) or 0 nM insulin (0 hour control). The control and treated adipocytes were lysed and the mRNAs were extracted for cDNA synthesis. Quantitative real-time PCR was used to measure expression of MINOR (FIG. 3A) and TR3 (FIG. 3B) genes. Results represent the mean±SE from three separate experiments. FIG. 3C shows inhibition of various signaling pathways decreases insulin-stimulated MINOR and TR3 gene expression. Fully differentiated 3T3-L1 adipocytes were treated with 100 nM of insulin alone (control) or plus LY294002 (PI 3-kinase inhibitor), SB203580 (p38 MAP kinase inhibitor), Ro318220 (protein kinase C inhibitor) for one hour. The control and treated adipocytes were lysed and the mRNAs were extracted for cDNAs synthesis. A quantitative real-time PCR was performed for detecting the expression levels of MINOR and TR3 genes. Results represent the mean±SE from three separate experiments.

FIGS. 4A-4D shows thiazolidinediones (TZDs) stimulate MINOR and TR3 gene expression in 3T3-L1 adipocytes. Fully differentiated 3T3-L1 adipocytes were treated with 10 uM of the indicated thiazolidinedione (troglitazone or pioglizatone) from 0 to 48 hours. Control cells received vehicle alone. The control and treated adipocytes were lysed and the mRNAs were extracted for cDNA synthesis. Quantitative real-time PCR was used to measure expression levels of MINOR (FIGS. 4A and 4B) and TR3 (FIGS. 4C and 4D) genes. Results represent the mean±SE from three separate experiments.

FIG. 5 shows MINOR enhances insulin-responsive glucose transport. Fully-differentiated adipocytes, a control adipocyte cell line hyperexpressing LacZ (LacZ18), and five different MINOR hyperexpressing cell lines (Minor; Minor2; MinorL1; MinorL2; and MinorL3) were incubated in the absence (basal) and presence of insulin (100 nM) for 30 min at 37° C. Measurements of 2-deoxy glucose transport were then performed (25). Results represent the mean±SE from three separate experiments; p<0.01 for comparing insulin-stimulated control and insulin stimulated MINOR hyperexpressing cells.

FIGS. 6A-6D show the effect of MINOR gene expression on insulin-mediated recruitment of GLUT4 glucose transporters to plasma membrane. MINOR gene transduced 3T3-L1 fibroblasts and control LacZ transduced fibroblasts were grown on glass cover slips and differentiated into adipocytes. Adipocytes were then stimulated for 30 min with (insulin-stimulated) or without (basal) 100 nM of insulin. The adipocytes were then washed, and disrupted by sonication leaving and plasma membrane sheets attached to cover slips (plasma membrane lawn assay) (26). Plasma membrane associated GLUT4 was detected using a polyclonal anti-GLUT4 antibody and a FITC-conjugated secondary antibody. (A) basal LacZ expressing adipocytes; (B) insulin-stimulated LacZ expressing adipocytes; (C) basal Minor expressing adipocytes; (D) insulin-stimulated Minor expressing adipocytes.

### DETAILED DESCRIPTION

The present disclosure describes the discovery of two novel candidate targets for pharmaceutical intervention from an exhaustive genomic study of over 100 skeletal muscle biopsies from normal and insulin resistant human donors. By assaying gene expression profiles for over 12,000 human sequence probes (Affymetrix U95A) followed by multivariate analysis of over 50 detailed clinical parameters for each biopsy (e.g., whole body glucose uptake assessed by hyperinsulinemic/euglycemic clamp), several hundred genes were identified with expression profiles which differ significantly in normal donors compared to insulin resistant individuals. Among this set are two genes (MINOR and TR3) in particular which, based on their protein sequence and presumed three-dimensional structure, are novel members of a sub-family of nuclear hormone receptors (NHRs) that include PPARγ, a known drug target of the thiozolidinediones (TZDs) class of insulin-sensitizing drugs (e.g., troglitazone, rosiglitazone, pioglitazone etc.). Many NHRs are known to be ‘master regulator’ proteins which control entire programs of downstream gene expression with consequent effects on tissue physiology. The TZDs represent a new class of anti-diabetic compounds and the only existing drugs focusing on insulin resistance in skeletal muscle. Though generally efficacious, these compounds are not without unwanted side-effects such as inducing weight gain in some patients. The discovery of novel drug targets with related biophysical properties but distinct function thus offers significant therapeutic potential. Moreover, the expression profile of the protein products of the MINOR and/or TR3 genes described in this disclosure may represent a biomarker used to assess the degree of insulin resistance in an individual, either through direct assay of skeletal muscle or indirectly by measuring the amount of a surrogate biomarker in blood which is itself regulated directly or indirectly by the expression profile of these NHRs.

The present disclosure demonstrates that the MINOR and TR3 genes are insulin responsive genes and that the MINOR and TR3 genes are differentially expressed as a function of insulin resistance and Type 2 Diabetes in humans and a variety of well characterized animal models. Furthermore, the present disclosure shows that MINOR and TR3 have a functional role in increasing insulin sensitivity. Specifically, the present disclosure demonstrates that: 1) MINOR expression is limited to insulin target tissues, muscle and fat, while TR3 is also expressed in these tissues as well as being more ubiquitously expressed; 2) the expression of the MINOR and TR3 genes are consistently decreased in muscle from rodent models of insulin resistance, Type 2 Diabetes, and obesity; 3) MINOR and TR3 are induced by insulin in 3T3-L1 adipocytes via signal transmission through metabolic pathways used in insulin-mediated signal transduction (PI3-kinase, p38MAP kinase and PKC); 4) MINOR and TR3 are induced by thiazolidinedione insulin-sensitizing drugs in 3T3-L1 adipocytes; 5) in lentiviral vector stably-transduced adipocyte cell lines, MINOR expression markedly augments insulin sensitivity for stimulation of glucose transport and 6) increased MINOR expression leads to increased stimulation of glucose transport as a result of increased mobilization of the GLUT4 glucose transporter proteins to the cell surface of insulin-responsive cell types.

Definitions

The terms “prevention”, “prevent”, “preventing”, “suppression”, “suppress” and “suppressing” as used herein refer to a course of action (such as administering a compound or pharmaceutical composition) initiated prior to the onset of a clinical symptom of a disease state or condition so as to prevent or reduce a clinical manifestation of the disease state or condition. Such preventing and suppressing need not be absolute to be useful.

The terms “treatment”, “treat” and “treating” as used herein refers a course of action (such as administering a compound or pharmaceutical composition) initiated after the onset of a clinical symptom of a disease state or condition so as to eliminate or reduce a clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method or compound of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method or compound of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as a part of a pharmaceutical composition, that is capable of having any detectable, positive effect on any symptom, aspect, or characteristics of a disease state or condition. Such effect need not be absolute to be beneficial.

The term “insulin resistance” as used herein refers to a condition where a normal amount of insulin is unable to produce a normal physiological or molecular response. In some cases, a hyper-physiological amount of insulin (such as over 100 units), either endogenously produced or exogenously added, is able to overcome the insulin resistance in whole or in part and produce a biologic response.

MINOR and TR3

The MINOR and TR3 genes are components of a general pathway that is involved in the insulin-mediated uptake of glucose from the blood. Consistent with this observation, the present disclosure shows that insulin increases the expression of the MINOR and TR3 genes and further shows that increased expression of the MINOR gene enhances insulin-responsive glucose transport and enhances insulin-mediated recruitment of the GLUT-4 glucose transporters to the plasma membrane. The present disclosure also provides for methods to treat and/or prevent insulin resistance in a subject in need of such treatment or prevention by activating the MINOR and/or TR3 pathway. The present disclosure provides for methods to treat or prevent disease states and conditions characterized by insulin resistance in a subject in need of such treatment or prevention by activating the MINOR and/or TR3 pathway. The disease states and conditions include, but are not limited to, metabolic syndrome, pre-diabetes, polycystic ovary syndrome, type 2 diabetes, dyslipidemia, obesity, infertility, inflammatory disorders, cancer, inflammatory diseases, Alzheimer's disease, hypertension, atherosclerosis, cardiovascular disease and peripheral vascular disease. Activating the MINOR and/or TR3 pathway includes, but is not limited to, increasing the absolute levels of the products of MINOR and/or TR3 gene expression, increasing the expression level or rate of the MINOR and/or TR3 genes, increasing the stability of the products of MINOR and/or TR3 gene expression, altering the levels and/or activity the polypeptide products of the MINOR and/or TR3 genes, increasing or decreasing the activity or expression of proteins downstream of MINOR and/or TR3 in the MINOR and/or TR3 pathway or a combination of any of the foregoing. Such activation of the MINOR and/or TR3 pathways may be achieved by direct or indirect methods. For example, direct methods may include, but are not limited to, providing increased levels of MINOR and/or TR3 gene expression, providing increased levels of the polypeptide products of MINOR and/or TR3 gene expression, or increasing the stability of the MINOR and/or TR3 genes or their expression products. Indirect methods may include, but are not limited to, activating downstream events in the MINOR and/or TR3 pathway. Such downstream events, include, but are not limited to, the stimulation or inhibition of proteins or enzymes that are influenced by MINOR and/or TR3. These proteins or enzymes may be involved in mediating the biological responses of the MINOR and/or TR3 pathway or antagonizing/down-regulating the biological responses of the MINOR and/or TR3 pathway. Any of the above may be accomplished through the administration of a pharmaceutical composition comprising at least one active ingredient or biologic or via the artificial induction of MINOR and/or TR3 genes or the expression products of such genes.

In one embodiment, the teachings of the present disclosure provide for the treatment of insulin resistance in a subject in need of such treatment. The method of treatment comprises the steps of identifying a subject in need of such treatment and activating the MINOR and/or TR3 gene pathway. In one embodiment, said activation is accomplished by increasing the expression of the MINOR and/or TR3 genes. In one embodiment, such increased expression is accomplished by administering a compound or pharmaceutical composition containing at least one active ingredient capable of activating the MINOR and/or TR3 pathway. In an alternate embodiment, such increased expression may be accomplished by introducing at least a portion of the MINOR and/or TR3 genes into a tissue (such as but not limited to skeletal muscle or adipose tissue) of said subject. Such activation would thereby treat insulin resistance in said subject. Such treatment may comprise increasing glucose uptake in insulin responsive tissues, increasing insulin sensitivity in insulin responsive tissues, or a combination of the foregoing. Other mechanisms may also be involved in such treatment.

In an alternate embodiment, the teachings of the present disclosure provide for the prevention of insulin resistance in a subject in need of such prevention. The method of prevention comprises the steps of identifying a subject in need of such prevention and activating the MINOR and/or TR3 gene pathway. In one embodiment, said activation is accomplished by increasing the expression of the MINOR and/or TR3 genes. In one embodiment, such increased expression is accomplished by administering a compound or pharmaceutical composition containing at least one active ingredient capable of activating the MINOR and/or TR3 pathway. In an alternate embodiment, such increased expression may be accomplished by introducing at least a portion of the MINOR and/or TR3 genes into a tissue (such as but not limited to skeletal muscle or adipose tissue) of said subject. Such activation would thereby prevent insulin resistance in said subject. Such prevention may comprise increasing glucose uptake in insulin responsive tissues, increasing insulin sensitivity in insulin responsive tissues, or a combination of the foregoing. Other mechanisms may also be involved in such prevention.

The present disclosure also provides for the treatment of disease states and conditions characterized by insulin resistance in a subject in need of such treatment by activating the MINOR and/or TR3 pathway. The method of treatment comprises the steps of identifying a subject in need of such treatment and activating the MINOR and/or TR3 gene pathway. In one embodiment, said activation is accomplished by increasing the expression of the MINOR and/or TR3 genes. In one embodiment, such increased expression is accomplished by administering a compound or pharmaceutical composition containing at least one active ingredient capable of activating the MINOR and/or TR3 pathway. In an alternate embodiment, such increased expression may be accomplished by introducing at least a portion of the MINOR and/or TR3 genes into a tissue (such as but not limited to skeletal muscle or adipose tissue) of said subject. Such activation would thereby treat at least one aspect of the disease state or condition in said subject. Such treatment may comprise increasing glucose uptake in insulin responsive tissues, increasing insulin sensitivity in insulin responsive tissues, or a combination of the foregoing. Other mechanisms may also be involved in such treatment.

The present disclosure also provides for the prevention of disease states and conditions characterized by insulin resistance in a subject in need of such treatment by activating the MINOR and/or TR3 pathway. The method of prevention comprises the steps of identifying a subject in need of such prevention and activating the MINOR and/or TR3 gene pathway. In one embodiment, said activation is accomplished by increasing the expression of the MINOR and/or TR3 genes. In one embodiment, such increased expression is accomplished by administering a compound or pharmaceutical composition containing at least one active ingredient capable of activating the MINOR and/or TR3 pathway. In an alternate embodiment, such activation may be accomplished by introducing at least a portion of the MINOR and/or TR3 genes into a tissue (such as but not limited to skeletal muscle or adipose tissue) of said subject. Such activation would thereby prevent at least one aspect of the disease state or condition in said subject. Such prevention may comprise increasing glucose uptake in insulin responsive tissues, increasing insulin sensitivity in insulin responsive tissues, or a combination of the foregoing. Other mechanisms may also be involved in such prevention

The methods of the treating and preventing discussed herein may also comprise further administering of one or more additional therapeutic agents agent in combination with those compounds or pharmaceutical compositions activating the MINOR and/or TR3 pathway. In one embodiment, the one or more additional therapeutic agents comprise metformin, sulforylurea or insulin.

Furthermore, the teachings of the present disclosure can be used to identify compounds that activate the MINOR and/or TR3 pathway. The compounds identified may thus be useful in the treatment and/or prevention methods described above. Such compounds may be small-molecule pharmaceuticals, peptides, biologics, various non-coding RNAs, antisense molecules and antibodies. The methods or assays for identifying such compounds comprise providing a cell line expressing at least a portion of the MINOR and/or TR3 genes, incubating said cells with a candidate compound and measuring a response to said candidate compound. Such a response may be any response that is measurable using analytical techniques currently known in the art. Exemplary responses include, but are not limited to, an increase in MINOR and/or TR3 gene expression levels, an increase in the level or activity of the polypeptides encoded by the MINOR and/or TR3 gene products or a functional response mediated by the MINOR and/or TR3 genes or their polypeptide products, such as increased glucose uptake, increased expression of a transporter involved in glucose regulation (such as, but not limited to, the GLUT4 transporter), increased translocation of a transporter involved in glucose regulation (such as, but not limited to, the GLUT4 transporter) to the cell membrane or increased activity of downstream signal transduction pathways regulated by MINOR and/or TR3 activity. Alternatively, cell lines may be provided that express downstream components activated in the MINOR and/or TR3 pathway and used in the methods of identification as discussed above. In addition, the response to said candidate compound measured may be a decrease in any of the criteria discussed above.

In an alternate embodiment, the teachings of the present disclosure can be used to identify compounds that bind to the expressed MINOR polypeptide, the expressed TR3 polypeptide or fragments of either polypeptide. The binding may be direct or indirect. By direct binding, it is meant the compound binds to the MINOR and/or TR3 polypeptide directly, without the involvement of an intermediate protein. By indirect binding, it is meant the compound binds to MINOR and/or TR3 via an intermediate polypeptide or structure, or the compound binds to a multi-protein complex comprising MINOR and/or TR3. The compounds identified may thus be useful in the treatment and/or prevention methods described above. Such compounds may be small-molecule pharmaceuticals, peptides, biologics, various non-coding RNAs, antisense molecules and antibodies. The methods or assays for identifying such compounds comprise providing a cell line expressing at least a portion of the MINOR and/or TR3 genes, incubating said cells with a candidate compound and measuring a determining whether said compound binds to the MINOR and/or TR3 polypeptides expressed in said cell line.

MINOR and/or TR3 genes, or fragments of these genes, may be also used utilizing the teachings of this invention for gene therapy by introducing the desired gene into the body of the patient who has or is suspected of having insulin resistance or a disease state or condition related to insulin resistance and is therefore in need of treatment and/or prevention. The desired gene may then be expressed and the treatment and/or prevention accomplished. Many methods exist for the introduction of genes or fragments thereof into a patient. For example, the gene may be introduced into a vector and introduced into the patient such that the gene or fragment thereof is expressed and the therapeutic potential realized. Exemplary methods of introduction include, but are not limited to, viral vectors (including retroviruses) and liposomes. Vectors may be introduced into a patient either in vivo or ex vivo. In the case of an in vivo treatment, the vector may be simply injected into the patient, for example parenterally, and allowed to find suitable target cells for gene expression. In the case of ex vivo treatment, cells are grown in vitro and transduced or transfected with the virus, embedded in a carrier such as a collagen matrix, which is then implanted in the patient, for example as a sub-cutaneous implant.

Pharmaceutical Compositions

The compound or pharmaceutical composition for use in the methods described may be formulated by any method known in the art. Certain exemplary methods for preparing the compounds and pharmaceutical compositions are described herein and should not be considered as limiting examples. Furthermore, the compounds or pharmaceutical compositions may be administered to the subject as is known in the art and determined by a healthcare provider. Certain modes of administration are provided herein and should not be considered as limiting examples. Furthermore, the compound or pharmaceutical composition may be administered with other agents in the methods described herein. Such other agents may be agents that increase the activity of the compounds disclosed, such as by limiting the degradation or inactivation of the compounds disclosed or increasing the absorption or activity of the compounds disclosed.

The compounds and pharmaceutical compositions described can be used in the form of a medicinal preparation, for example, in aerosol, solid, semi-solid or liquid form which contains the compounds disclosed as an active ingredient. In addition, the pharmaceutical compositions may be used in an admixture with an appropriate pharmaceutically acceptable carrier. Such pharmaceutically acceptable carriers include, but are not limited to, organic or inorganic carriers, excipients or diluents suitable for pharmaceutical applications. The active ingredient may be compounded, for example, with the usual non-toxic pharmaceutically acceptable carriers, excipients or diluents for tablets, pellets, capsules, inhalants, suppositories, solutions, emulsions, suspensions, aerosols and any other form suitable for use. Pharmaceutically acceptable carriers for use in pharmaceutical compositions are well known in the pharmaceutical field, and are described, for example, in Remington: The Science and Practice of Pharmacy Pharmaceutical Sciences, Lippincott Williams and Wilkins (A. R. Gennaro editor, 20^{th} edition). Such materials are nontoxic to the recipients at the dosages and concentrations employed and include, but are not limited to, water, talc, gum acacia, gelatin, magnesium trisilicate, keratin, colloidal silica, urea, buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, lactose, mannitol, glucose, mannose, dextrins, potato or corn starch or starch paste, chelating agents such as EDTA, sugar alcohols such as mannitol or sorbitol, counterions such as sodium and/or nonionic surfactants such as Tween, Pluronics or polyethyleneglycol. In addition, the pharmaceutical compositions may comprise auxiliary agents, such as, but not limited to, taste-enhancing agents, stabilizing agents, thickening agents, coloring agents and perfumes.

Pharmaceutical compositions may be prepared for storage or administration by mixing a compound of the present disclosure having a desired degree of purity with physiologically acceptable carriers, excipients, stabilizers, auxiliary agents etc. as is known in the pharmaceutical field. Such pharmaceutical compositions may be provided in sustained release or timed release formulations.

The pharmaceutical compositions may be administered orally in solid dosage forms, such as capsules, tablets, and powders, or in liquid dosage forms, such as elixirs, syrups and suspensions. It can also be administered parenterally, in sterile liquid dosage forms. Furthermore, pharmaceutical compositions may be administered parenterally by transmucosal delivery via solid, liquid or aerosol forms of transdermally via a patch mechanism or ointment. Various types of transmucosal administration include respiratory tract mucosal administration, nasal mucosal administration, oral transmucosal (such as sublingual and buccal) administration and rectal transmucosal administration.

For preparing solid compositions such as, but not limited to, tablets or capsules, the pharmaceutical compositions may be mixed with an appropriate pharmaceutically acceptable carriers, such as conventional tableting ingredients (lactose, sucrose, mannitol, corn starch, potato starch, alginic acid, microcrystalline cellulose, acacia, gelatin, gums, colloidal silicon dioxide, croscarmellose sodium, talc, sorbitol, stearic acid magnesium stearate, calcium stearate, zinc stearate, stearic acid, dicalcium phosphate other excipients, colorants, diluents, buffering agents, disintegrating agents, moistening agents, preservatives, flavoring agents, and pharmacologically compatible carriers) and diluents (including, but not limited to, water, saline or buffering solutions) to form a substantially homogenous composition. The substantially homogenous composition means the components (a compound as described herein and a pharmaceutically acceptable carrier) are dispersed evenly throughout the composition so that the composition may be readily subdivided into equally effective unit dosage forms such as tablets, pills and capsules. The solid compositions described may be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact through the stomach or to be delayed in release. A variety of materials can be used for such enteric layers or coatings such materials including a number of polymeric acids and mixtures of polymeric acids with such materials as shellac, cetyl alcohol and cellulose acetate. The active compounds may also be formulated in rectal compositions such as suppositories or retention enemas, e.g., containing conventional suppository bases such as cocoa butter or other glycerides. The solid compositions may also comprise a capsule, such as hard- or soft-shelled gelatin type containing, for example, surfactants, lubricants, and inert fillers, such as lactose, sucrose, calcium phosphate, and corn starch.

For intranasal administration, intrapulmonary administration or administration by other modes of inhalation, the pharmaceutical compositions may be delivered in the form of a solution or suspension from a pump spray container or as an aerosol spray presentation from a pressurized container or nebulizer, with the use of a suitable propellant (e.g., dichlorodifluoromethane, trichlorofluoromethane, dichlorotetrafluoroethane, nitrogen, propane, carbon dioxide or other suitable gas) or as a dry powder. In the case of an aerosol or dry powder format, the amount (dose) of the compound delivered may be determined by providing a valve to deliver a metered amount.

Liquid forms may be administered orally, parenterally or via transmucosal administration. Suitable forms for liquid administration include aqueous solutions, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. For buccal or sublingual administration, the composition may take the form of tablets or lozenges formulated in conventional manners. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds disclosed (whether alone or in pharmaceutical compositions) may be formulated for parenteral administration. Parenteral administration includes, but is not limited to, intravenous administration, subcutaneous administration, intramuscular administration, intradermal administration, intrathecal administration, intraarticular administration, intracardiac administration, retrobulbar administration and administration via implants, such as sustained release implants.

The pharmaceutical compositions may be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

The pharmaceutical compositions are administered in pharmaceutically effective amount. The pharmaceutically effective amount will, of course, vary depending upon known factors, such as the pharmacodynamic characteristics of the particular compound and its mode and route of administration; the age, health and weight of the subject; the severity and stage of the disease state or condition; the kind of concurrent treatment; the frequency of treatment; and the effect desired. The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

### EXAMPLES

MINOR and TR3 Genes are Differentially Expressed in Insulin Resistance

The primary target tissues for insulin action are skeletal muscle, adipose tissue, and the liver tissue. Skeletal muscle is the predominant target tissue for insulin-mediated glucose uptake (responsible for approximately 95% of glucose uptake), and is a critical locus of insulin resistance. Defects in glucose uptake in skeletal muscle is a predominate contributor to the clinical manifestations of insulin resistance. The molecular basis for insulin resistance is not well understood, but appears to involve defects in insulin signal transduction and abnormal cellular trafficking of glucose transporter proteins (5). To better understand the molecular defects responsible for human insulin resistance, skeletal muscle biopsies were obtained from volunteers in three metabolically-defined subgroups: A) insulin sensitive: (B) insulin resistant; and (C) type 2 diabetic. Biopsies from each subgroup were obtained under both basal conditions and after three hours of hyperinsulinemia. Differential gene expression in these muscle biopsies was assessed using cDNA microarray technology utilizing the Affymetrix Hu95A gene expression chips (carried out as per manufacturer's instructions). The effect of insulin on gene expression in human muscle has not been extensively studied, and most prior studies focused on single genes or small numbers of genes with a limited focus (6-8).

We found that insulin resistance in subjects with or without Type 2 diabetes is associated with differential gene expression in skeletal muscle when compared with insulin sensitive individuals. In particular, over 100 genes coding for transcription factors were shown to be acutely regulated by insulin, and/or differentially expressed in skeletal muscle among the three defined subgroups. Many of these transcription factors expressed the zinc finger motif. Table 1 shows data (i.e., p values for statistically significant differential expression) for two of these genes including MINOR (Mitogen-Inducible Nuclear Orphan Receptor; GenBank accession number: U12767; SEQ ID NO. 1 shows the nucleotide sequence of the MINOR gene, designating the CDS; SEQ ID NO. 2 shows the polypeptide sequence of the peptide encoded by the MINOR gene) and TR3 (GenBank accession number: L13740; SEQ ID NO. 3 shows the nucleotide sequence of the TR3 gene, designating the CDS; SEQ ID NO. 4 shows the polypeptide sequence of the peptide encoded by the TR3 gene). Both MINOR and TR3 belong to the NGFI-B family of orphan nuclear receptors.

MINOR (also known as NOR-1, TEC, and CHN and designated NR4A3 in the nuclear receptor nomenclature system) is an orphan nuclear receptor originally identified as a protein induced in primary cultures of rat embryonic forebrain neurons undergoing apoptosis (9). Homology analysis of its DNA binding domain identifies MINOR as a member of the NGFI-B family of orphan nuclear receptors together with Nurr1 (also known as TINUR, NOT, and designated NR4A2 in the nuclear receptor nomenclature system) and TR3 (also known as Nur77, NGFI-B, and designated NR4A1 in the nuclear receptor nomenclature system). The NGFI-B receptors are members of steroid/thyroid receptor superfamily.

Among receptors in the NGFI-B family, homologies in the N-terminal transactivation domains and the C-terminal “ligand binding domains” are 37-53% and 53-77%, respectively. In the absence of any ligand, MINOR, Nurr1 and TR3 can each bind and activate the NGFI-B-responsive DNA element characterized by the nucleotide sequence AAAGGTCA (10). MINOR, Nurr1 and TR3 each share greater that 97% homology in their DNA binding domains, which consist of two zinc fingers and a domain termed the A box. In the presence of retinoic acid, however, TR3 and Nurr1, but not MINOR, can heterodimerize with the retinoid X receptor and regulate a DNA element composed of direct repeats separated by five nucleotides (DR5) (11-13). The NGFI-B proteins are immediate early gene products which are involved in neuroendocrine regulation, neural differentiation, liver regeneration, cell apoptosis, and mitogenic stimulation in different cell types (14-17).

Tissue Specific Expression

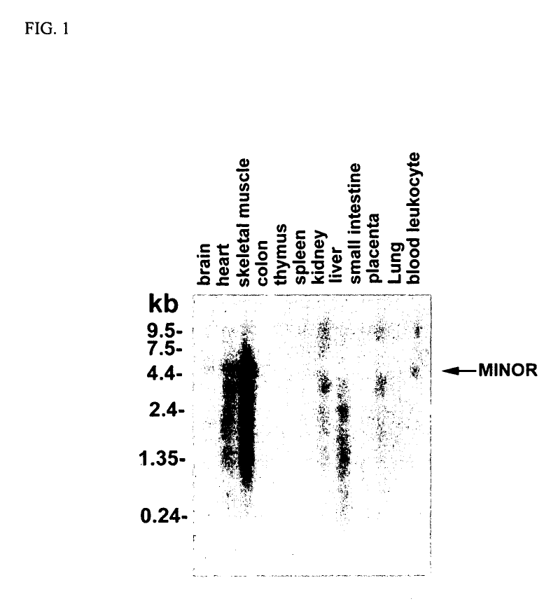

To determine whether MINOR and/or TR3 regulation plays a role in insulin resistance, the tissue specific expression of MINOR and TR3 genes was examined. Northern blot analysis was performed to examine MINOR and TR3 gene expression in brain, heart, skeletal muscle, colon, thymus, spleen, kidney, liver, small intestine, placenta, lung and blood leukocytes (FIG. 1). Human multiple tissue northern blot was purchased from Clontech (Palo Alto, Calif.). Each of the lanes contained mRNA from the designated human tissue and the amount of each RNA blotted on the membrane was normalized with a β-actin cDNA control probe. The probe for detecting MINOR gene hybridization was a 1.1 kb cDNA fragment (17). The results show that MINOR was highly expressed only in human skeletal muscle and adipocytes, both of which comprise classic insulin target tissues. In contrast, TR3 was expressed in skeletal muscle and adipocytes, as well as multiple other tissues (data not shown).

MINOR and TR3 Expression are Reduced in Skeletal Muscles of Diabetic Rats and Mice

In order to determine whether the MINOR and/or TR3 genes are abnormally regulated in insulin resistance and/or Type 2 Diabetes, MINOR and TR3 gene expression was analyzed in several animal models. Zucker diabetic fatty (ZDF) rats, streptozotocin (STZ)-induced diabetic rats, db/db mice (mice that express defects in leptin receptor), and ob/ob mice (mice that express defects in the adipocyte-derived hormone leptin) and are well characterized as insulin-resistant, diabetic or obese animal models (18, 20). Skeletal muscle tissue from these diabetic and insulin resistant rats or mice was obtained, homogenized and the mRNA extracted for cDNA synthesis. The cDNA was used in quantitative real-time PCR to measure the expression of MINOR and TR3 genes.

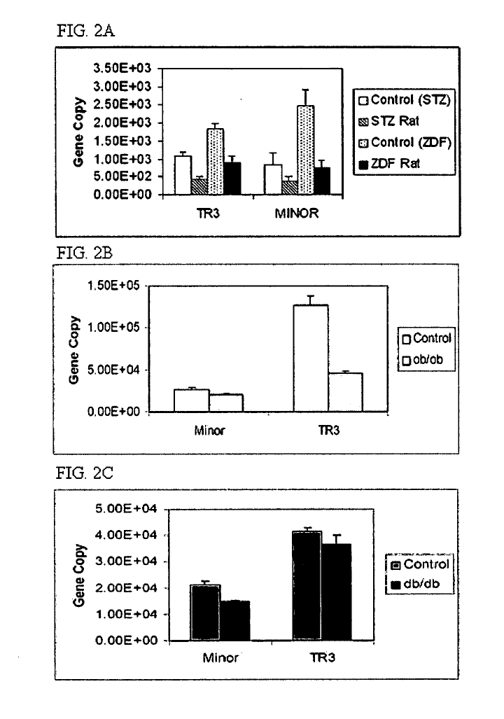

The expression of MINOR and TR3 genes in skeletal muscle samples showed a decrease in MINOR and TR3 gene expression in these animal models of insulin resistance, diabetes and obesity as compared to the appropriate control animals. FIG. 2A shows that MINOR and TR3 gene expression was significantly decreased in the STZ and ZDF rat models. FIGS. 2B and 2C show similar results for the ob/ob and db/db mice models. Therefore, decreased expression of the MINOR and TR3 genes, which code for two transcription factors, is consistently associated with insulin resistance, diabetes and obesity in animal models. This result correlates with the decreased expression of MINOR and TR3 genes observed in skeletal muscle biopsies from insulin resistant and type-2 diabetic human subjects (Table 1).

The Expression of MINOR and TR3 in 3T3-L1 Adipocytes

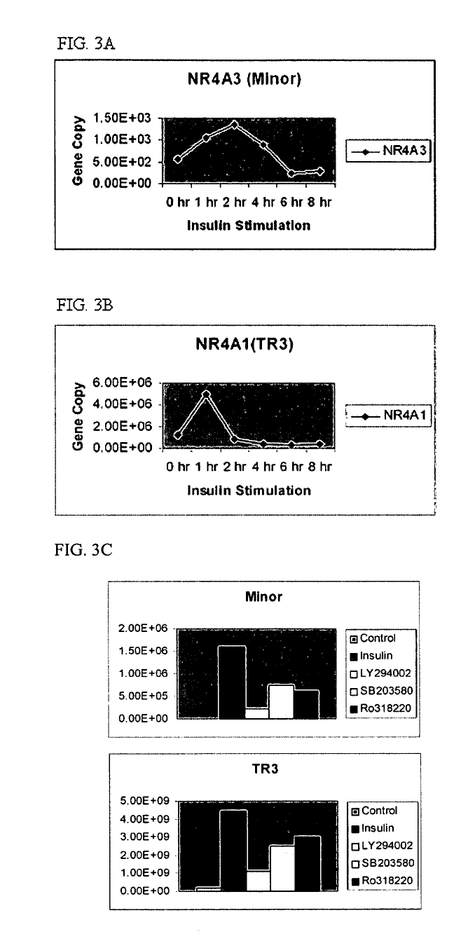

Since MINOR and TR3 gene expression was strongly induced by insulin stimulation in human skeletal muscles in the microarray analysis described above (Table 1), the responsiveness of MINOR and TR3 gene expression to insulin stimulation was examined in adipocytes, another major insulin target tissue. Fully differentiated 3T3-L1 adipocytes were treated with 100 nM of insulin for various times (0 to 8 hours; the 0 hour time point served as the control and received no added insulin). The control and treated adipocytes were lysed and the mRNAs were extracted for cDNAs synthesis. A quantitative real-time PCR was performed for detecting the expression levels of MINOR and TR3 genes. As shown in FIGS. 3A and 3B, insulin stimulated MINOR and TR3 gene expression (Nurr1 gene expression was not stimulated; data not shown) in fully differentiated adipocytes. FIG. 3A shows the effect of insulin on MINOR gene expression in adipocytes and shows a rapid increase in MINOR gene expression that is sustained over a 4 hour period. Insulin also stimulated TR3 gene expression adipocytes, but with a different temporal response with TR3 expression levels returning to baseline by hour 2 after treatment (FIG. 3B).

The stimulatory effect of insulin on MINOR and TR3 gene expression was dependent on signal transduction via the PI-3-kinase pathway, the p38 MAP-K pathway and the protein kinase C (PKC) pathway. Blocking any of these pathways by administration of specific chemical inhibitors decreased the ability of insulin to stimulate MINOR or TR3 gene expression (FIG. 3C). Fully differentiated 3T3-L1 adipocytes were treated for 1 hour with 100 nM of insulin alone or 100 nM of insulin plus LY294002 (PI 3-kinase inhibitor, 10 nM), SB203580 (p38 MAP kinase inhibitor, 10 nM), or Ro318220 (protein kinase C inhibitor, 50 nM). Control adipocytes received no insulin or inhibitors. The control and treated adipocytes were lysed and the mRNAs were extracted for cDNAs synthesis. A quantitative real-time PCR was performed for detecting the expression levels of MINOR and TR3 genes. As can be seen in FIG. 3C, inhibiting the PI-3-kinase pathway, the p38 MAP-K pathway or the protein kinase C (PKC) pathway inhibited the ability of insulin to stimulate MINOR and TR3 gene expression. Importantly, each of these pathways are implicated in the ability of insulin to stimulate the regulation and cycling of the GLUT4 glucose transporter. These results show that MINOR and TR3 are insulin responsive genes and are expressed in a specific target of insulin action (i.e. adipocytes). Furthermore, the results show that the insulin responsive increase in gene expression can be blocked, at least partially, with well characterized inhibitors of the insulin signal transduction cascade.

Thiazolidinediones (TZDs) Induce MINOR and TR3 in 3T3-L1 Adipocvtes

Next, the effect of several drugs in the thiazolidinedione class of insulin sensitizing drugs, namely troglitazone and pioglitazone, were examined for their ability to induce MINOR and TR3 gene expression in 3T3-L1 adipocytes. The thiazolidinediones (TZD) and thiazolidinedione-like drugs are known to improve insulin resistance and are used as a treatment for diabetes in animal models and patients (22). These drugs reduce plasma glucose and concomitantly lower hyperinsulinemia by improving the stimulation of muscle glucose uptake and the inhibition of hepatic glucose production by insulin. The antidiabetic actions of TZDs and TZD-like drugs are due to the activation of the peroxisome proliferator-activated receptor gamma (PPARγ) (23), a member of the nuclear receptor superfamily of transcription factors (24). TZD agonists of PPARγ have an effect on glucose disposal which is typically thought to occur in the muscle, however PPARγ is expressed in very small amounts in this tissue. Given the results that MINOR and TR3 are highly expressed in skeletal muscle, it is reasonable to speculate that TZDs could stimulate MINOR and TR3 gene expression. Alternatively, activation of MINOR and TR3 could represent downstream consequences of PPARγ activation, and constitute direct mediators of the insulin-sensitizing TZD drug effect.

The insulin sensitizing effects of TZDs and TZD-like drugs are accompanied by unwanted side effects such as weight gain, edema, intravascular volume expansion, anemia, and congestive heart failure. It remains possible that drugs acting downstream of PPARγ activation could lead to increased insulin sensitivity without the unwanted side effects of TZDs.

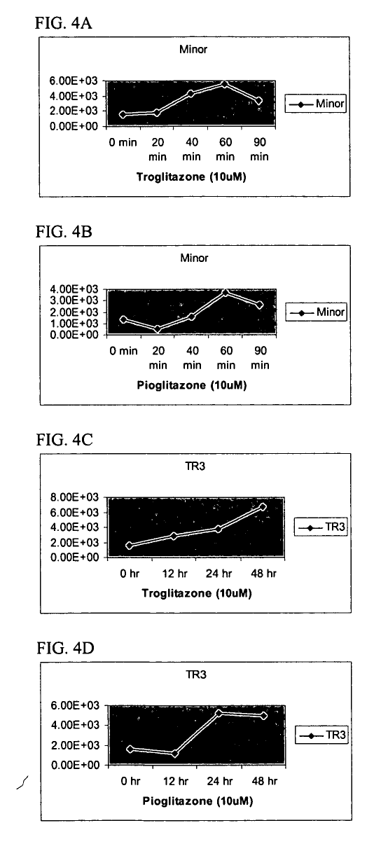

Fully differentiated 3T3-L1 adipocytes were treated with 10 uM of the indicated thiazolidinedione (troglitazone or pioglizatone) from 0 to 48 hours. Control cells (0 hour time point) received vehicle alone. The control and treated adipocytes were lysed and the mRNAs were extracted for cDNA synthesis. Quantitative real-time PCR was used to measure expression levels of MINOR (FIGS. 4A and 4B) and TR3 (FIGS. 4C and 4D) genes. FIGS. 4A-D show that troglitazone and pioglitazone stimulate MINOR and TR3 gene expression in adipocyte cells. Expression of these genes was unaffected in control cells over these time courses. Therefore, MINOR and TR3 were induced by thiazolidinedione drugs, which are used to increase insulin sensitivity in humans.

MINOR Enhances Insulin Action on Glucose Uptake in 3T3-L1 Adipocvtes

To identify the biological or functional effects of increased MINOR gene expression, recombinant MINOR lentivirus vectors were generated and used to stably transduce 3T3-L1 adipocyte cell lines. Several MINOR lentiviral vectors were generated. The MINOR cDNA sequences used are designated as follows: (i) MINOR; (ii) MINOR 2; (iii) MINOR L1; (iv) MINOR L2; and (v) MINOR L3. These constructs contained full-length MINOR coding sequences as follows: full CDS for MINOR; nucleotides 183 to 1971 of the CDS for MINOR 2; nucleotides 100 to 1971 of the CDS for MINOR L1, MINOR L2 and MINOR L3 (17). In each case, fusion cDNAs were created through the addition of a V5 epitope tag, and were cloned into a ViraPower-CMV vector (Invitrogen). The recombinant lentiviral plasmids and a control lentiviral LacZ gene construct were transfected into HEK293 cells to generate the recombinant lentiviruses. X-gal staining was performed to confirm that the HEK293 cell transfection was successful and that infectious virus particles were produced.

To establish stable 3T3-L1 adipocyte cell lines which overexpress the MINOR or LacZ genes, recombinant MINOR or LacZ lentiviral stocks purified from HEK293 cells were used to infect 3T3-L1 adipocytes. Forty-eight hours post-transduction, these cells were placed under blasticidin selection (10 μg/ml) for 20 days. The tests for stable recombinant MINOR or LacZ gene expression were performed after antibiotic selection by Western blot analyses. These experiments yielded multiple clonal adipocyte cell lines with sustained expression of the described MINOR gene constructs the or LacZ gene.

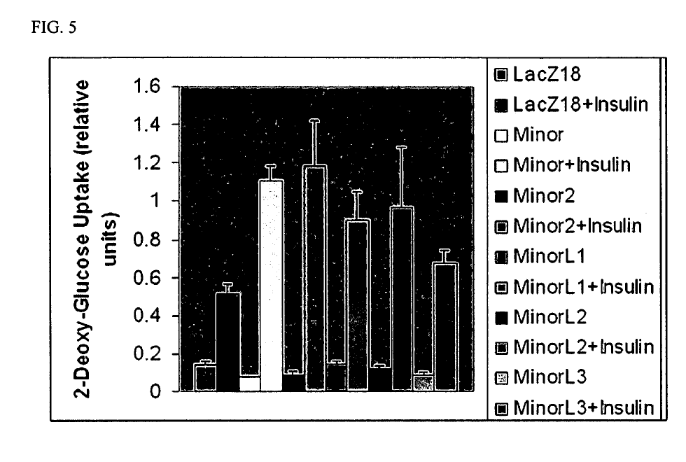

Glucose uptake is the rate-limiting step in insulin's ability to stimulate glucose uptake and metabolism. Insulin augments the transport of glucose into target cells by increasing the concentration of a specific glucose transporter isoform, GLUT4, at the cell surface (5, 21). If MINOR is involved in the regulation of GLUT4, then MINOR transduced adipocytes should display increased insulin responsiveness for stimulation of the glucose transport system. As shown in FIG. 5, insulin's ability to maximally stimulate glucose uptake in fully differentiated adipocytes was increased over 80% (P<0.01) in the MINOR overexpressing cells when compared with control LacZ overexpressing adipocytes. This was true for all 5 stably transduced MINOR-expressing cell lines. In full-dose response curves, glucose transport responses were markedly enhanced over the full range of insulin concentrations in MINOR-transduced cells, without changes in the insulin ED₅₀ for glucose transport stimulation (data not shown).

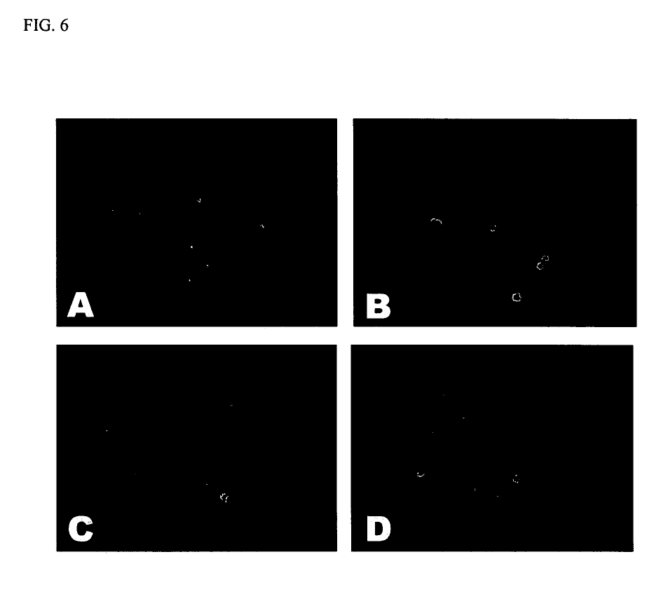

FIGS. 6A-6D show that MINOR gene expression increases the ability of insulin stimulation to recruit the GLUT-4 glucose transporter to the plasma membrane of adipocyte cells (SEQ ID NO. 5 shows the nucleotide sequence of the GLUT4 gene, designating the CDS; SEQ ID NO. 6 shows the polypeptide sequence of the peptide encoded by the GLUT4 gene). These experiments use the “plasma membrane lawn” technique (26) to quantify the relative amounts of GLUT4 glucose transporter proteins in the cell surface plasma membrane. MINOR gene transduced 3T3-L1 fibroblasts and control LacZ transduced fibroblasts were grown on glass cover slips and differentiated into adipocytes. In these experiments, the full-length MINOR constructs were used (similar results were obtained for the vectors containing MINOR nucleotides 183-1971 and 100-1971). Adipocytes were then stimulated for 30 min with or without (basal) 100 nM of insulin. The adipocytes were then washed, and disrupted by sonication leaving and plasma membrane sheets attached to cover slips. Plasma membrane associated GLUT4 was detected using a polyclonal anti-GLUT4 antibody and a FITC-conjugated secondary antibody.

In control (LacZ transfected) adipocytes, insulin stimulation leads to increased GLUT4 staining in the plasma membrane, consistent with the known ability of insulin to recruit increased numbers of intracellular GLUT4 transporters to the cell surface. In adipocytes hyperexpressing the MINOR gene, the ability of insulin stimulation to recruit GLUT4 glucose transporters to the plasma membrane is clearly stimulated as compared to control LacZ expressing adipocytes (compare FIGS. 6A and 6B to FIGS. 6C and 6D). Therefore, MINOR gene expression increases insulin-stimulated glucose transport by enhancing the normal cellular processes that mediate the ability of insulin stimulation to transport glucose from the blood and into tissue.

### Discussion of Examples

The above examples demonstrate that the MINOR and TR3 genes are insulin responsive genes and that the MINOR and TR3 genes are differentially expressed as a function of insulin resistance and Type 2 Diabetes in humans and a variety of well characterized animal models. Furthermore, the present disclosure shows that MINOR and TR3 have a functional role in increasing insulin sensitivity. Specifically, the examples demonstrate that: 1) MINOR expression is limited to insulin target tissues, muscle and fat, while TR3 is also expressed in these tissues as well as being more ubiquitously expressed; 2) the expression of the MINOR and TR3 genes are consistently decreased in muscle from rodent models of insulin resistance, Type 2 Diabetes, and obesity; 3) MINOR and TR3 are induced by insulin in 3T3-L1 adipocytes via signal transmission through metabolic pathways used in insulin-mediated signal transduction (PI3-kinase, p38MAP kinase and PKC); 4) MINOR and TR3 are induced by thiazolidinedione insulin-sensitizing drugs in 3T3-L1 adipocytes; 5) in lentiviral vector stably-transduced adipocyte cell lines, MINOR expression markedly augments insulin sensitivity for stimulation of glucose transport and 6) increased MINOR expression leads to increased stimulation of glucose transport as a result of increased mobilization of the GLUT4 glucose transporter proteins to the cell surface of insulin-responsive cell types.

These results demonstrate that insulin resistance is associated with reduced expression of MINOR and TR3 genes. Furthermore, the present disclosure demonstrates that the stimulation of MINOR and TR3 gene expression can produce a marked decrease insulin resistance and a corresponding increase in insulin sensitivity by enhancing the insulin-mediated clearance of glucose from the blood.

Therefore, MINOR and TR3 are attractive and novel therapeutic targets for combating insulin resistance. Specifically, activation of MINOR and TR3 pathway by small-molecule pharmaceuticals, by genetic manipulation, or by other means represents a new and effective approach to the treatment of insulin resistance or disease states or conditions characterized by insulin resistance. Furthermore, activation of downstream signaling events mediated by the MINOR and/or TR3 pathway is also a new and effective approach to the treatment of insulin resistance or disease states or conditions characterized by insulin resistance. These disease states and conditions include, but are not limited to, metabolic syndrome, pre-diabetes, polycystic ovary syndrome, type 2 diabetes, dyslipidemia, obesity, infertility, inflammatory disorders, cancer, inflammatory diseases, Alzheimer's disease, hypertension, atherosclerosis, cardiovascular disease and peripheral vascular disease.

As disclosed, in addition to constituting new direct therapeutic targets for the treatment and prevention of insulin resistance and the disease states and conditions characterized by insulin resistance, MINOR and TR3 can be also utilized for screening new therapeutic agents for treating insulin resistance and disease states and conditions characterized by insulin resistance. Such new therapeutic agents include, but are not limited to, synthetic or endogenous ligands for MINOR and TR3. Similarly, MINOR and TR3 could provide the basis for identifying downstream molecular events that mediate desirable therapeutic effects. For example, a search for genes regulated by MINOR and TR3, or factors that interact with the corresponding encoded proteins, could identify new drug targets or therapeutic approaches. The advances in the understanding of the molecular pathways that underlie MINOR or TR3 expression, gene regulation by these transcription factors, and related cellular effects could potentially point to a number of new opportunities for therapeutic intervention.

The foregoing description illustrates and describes the compounds of the present disclosure. Additionally, the disclosure shows and describes only certain embodiments of the compounds but, as mentioned above, it is to be understood that the teachings of the present disclosure are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. All references cited herein are incorporated by reference as if fully set forth in this disclosure.

### REFERENCES

1. Reaven, G. M. (1988). Role of insulin resistance in human disease. Diabetes 37, 1595-1606.
2. Reaven, G. M. (1991). Insulin resistance, hyperinsulinemia, hypertriglyceridemia, and hypertension. Diabetes Care 14, 195-202.
3. DeFronzo, R. A., and Ferrannini, E. (1991). Insulin resistance: A multifaceted syndrome responsible for NIDDM, obesity, hypertension, dyslipidemia, and atherosclerotic cardiovascular disease. Diabetes Care 14, 173-194.
4. Garvey W T, Kwon S, Zheng D, Shaughnessy S, Wallace P, Pugh K, Jenkins A J, Klein R L, Liao Y. (2003). The Effects of Insulin Resistance and Type 2 Diabetes Mellitus on Lipoprotein Subclass Particle Size and Concentration Determined by Nuclear Magnetic Resonance. Diabetes, 52, 453-462.
5. Hunter, S. J., and Garvey, W. T. (1998). Insulin action and insulin resistance: diseases involving defects in insulin receptors, signal transduction, and the glucose transport effector system. American Journal of Medicine. 105:331-345.
6. O'Brien, R. M., and Granner, D. K. (1996). Regulation of gene expression by insulin. Physiol. Rev. 76, 1109-1161.
7. Laville, M., Auboeuf, D., Khalfallah, Y., Vega, N., Rou, J. P., and Vidal, H. (1996). Acute regulation by insulin of phosphatidylinositol-3-kinase, Rad, Glut 4, and lipoprotein lipase mRNA levels in human muscle. J. Clin. Invest. 98, 43-49.
8. Bao, S., Kennedy, A., Wojciechowski, B., Wallace, P., Ganaway, E., and Garvey, W. T. (1998). Expression of mRNAs encoding uncoupling proteins in human skeletal muscle: effects of obesity and diabetes. Diabetes 47, 1935-1940.
9. Ohkura, T., Hijikuro, M., Yamanoto, A., and Miki, K. (1994). Molecular cloning of a novel thyroid/steroid receptor superfamily gene from cultured rat neuronal cells. Biochem. Biophys. Res. Commun. 205, 1959-1965.
10. Wilson, T. E., Fahrner, T. J., Johnston, M., and Milbrandt, J. (1991). Identification of the DNA binding site for NGFI-B by genetic selection in Yeast. Science 252, 1296-1300.
11. Mangelsdorf, D. J., and Evans, R. M. (1995). The RXR heterodimers and orphan receptors. Cell 83, 841-850.
12. Perlmann, T., and Jansson, L. (1995). A novel pathway for vitamin A signaling mediated by RXR heterodimerization with NGFI-B and NURR1. Genes Dev. 9, 769-782.
13. Philips, A., Lesage, S., Gingras, R., Maira, M.-H., Gauthier, Y., Hugo, P., and Drouin, J. (1997). Novel dimeric Nur77 signaling mechanism in endocrine and lymphoid cells. Mol. Cell. Biol. 17, 5946-5951.
14. Milbrandt J. (1988). Nerve growth factor induces a gene homologous to the glucocorticoid receptor gene. Neuron 1, 183-188.
15. Hazel T. G., Nathans D., Lau L. F. (1988). A gene inducible by serum growth factors encodes a member of the steroid and thyroid hormone receptor superfamily. Proc. Natl. Acad. Sci. USA 85, 8444-8448.
16. Scearce L. M., Laz T. M., Hazel T. G., Lau L. F., Taub R. (1993). RNR-1, a nuclear receptor in the NGFI-N/Nur77 family that is rapidly induced in regenerating liver. J. Biol. Chem. 268, 8855-8861.
17. Hedvat C. V., Irving S. G. (1995). The isolation and characterization of MINOR, a novel mitogen-induced nuclear orphan receptor. Mol. Endocrinol. 9, 1692-1700.
18. Tofovic S. P., and Jackson, E. K. (2003). Rat models of the metabolic syndrome. Methods in Molecular Medicine. 86, 29-46.
19. Tschop, M., and Heiman, M. L. (2001). Rodent obesity models: an overview. Experimental & Clinical Endocrinology & Diabetes. 109, 307-319.
20. Moore, G. B., Chapman, H., Holder, J. C., Lister, C. A., Piercy, V., Smith, S. A., and Clapham, J. C. (2001). Differential regulation of adipocytokine mRNAs by rosiglitazone in db/db mice. Biochemical & Biophysical Research Communications. 286, 735-741.
21. Hunter, S., and Garvey, W. T. (1998). Insulin action and insulin resistance: Diseases involving defects in insulin receptors, signal transduction, and the glucose transport effector system. Am. J. Med. 105, 331-345.
22. Saltiel, A. R., and Olefsky, J. M. (1996). Thiazolidinediones in the treatment of insulin resistance and type II diabetes. Diabetes 45, 1661-1669.
23. Lehmann, J. M., Moore, L. B., Smith-Oliver, T. A., Wilkinson, W. O., Wilson, T. M., and Kliewer, S. A. (1995). An antidiabetic thazolidinedione is a high affinity ligand for PPARγ. J. Biol. Chem.270, 12953-12956.
24. Braissant, O., Foufelle, F., Scotto, C., Dauca, M., and Wahli, W. (1996). Differential expression of peroxisome proliferators-activated receptors (PPARs): tissue distribution of PPAR-alpha, -beta and -gamma in the adult rat. Endocrinology 137, 354-366.
25. Mayor, P., Maianu, L., and Garvey, W. T. 1992. Glucose and insulin chronically regulate insulin action via different mechanisms in BC3H1 myocytes: Effects on glucose transporter gene expression. Diabetes 41: 274-285.

26. Nelson, B. A., Robinson, K. A., and Buse, M. G. 2000. High glucose and glucosamine induce insulin resistance via different mechanisms in 3T3-L1 adipocytes. Diabetes 49: 981-991.

TABLE 1
Microarray Results Comparing Gene Expression in Skeletal
Muscle Among Insulin Sensitive, Insulin Resistant, and Type 2
Diabetic Humans:
p values for statistically significant differential expression

| | | | | | | Insulin- | |
|---|---|---|---|---|---|---|---|
| | Basal | | | Basal | | Stimulated | |
| | vs | | | Tissues | | Tissues | |
| | Insulin Stimulation | | | IS vs | | IS vs | |
| | IS | IR | DM | IR | IS vs DB | IR | IS vs DB |
| MINOR | .0001 | .0548 | .0020 | .0053 | .0033 | .0023 | .0053 |
| TR3 | <.0001 | <.0001 | .0002 | NS | NS | .0105 | .0375 |

Note:
IS = insulin sensitive as assessed by clamp study and normoglycemic;
IR = insulin resistant as assessed by clamp study and normoglycemic;
DB = insulin-resistant patients with untreated Type 2 Diabetes.
NS = not significant.
Bold type indicates conditions being compared.

What is claimed:

1. A method for treating or preventing insulin resistance in a subject, said method comprising identifying a subject in need of said treatment or prevention and activating an insulin responsive gene selected from the group consisting of MINOR, TR3 and MINOR and TR3.

2. The method of claim 1 where said activating is accomplished by administering a compound which activates said insulin responsive gene.

3. The method of claim 1 where said activation is accomplished by introducing at least a fragment of said insulin responsive gene into a tissue of said subject such that the expression of said insulin responsive gene is increased.

4. The method of claim 3 where said tissue is skeletal muscle or adipose tissue.

5. The method of claim 1 further comprising administering at least one secondary therapeutic agent to said subject.

6. The method of claim 5 where said secondary therapeutic agent is selected from the group consisting of metformin, sulforylurea and insulin.

7. A method for treating or preventing a disease state associated insulin resistance in a subject, said method comprising identifying a subject in need of said treatment or prevention and activating an insulin responsive gene selected from the group consisting of MINOR, TR3 and MINOR and TR3.

8. The method of claim 7 where said activating is accomplished by administering a compound which activates said insulin responsive gene.

9. The method of claim 7 where said activation is accomplished by introducing at least a fragment of said insulin responsive gene into a tissue of said subject such that the expression of said insulin responsive gene is increased.

10. The method of claim 9 where said tissue is skeletal muscle or adipose tissue.

11. The method of claim 7 further comprising administering at least one secondary therapeutic agent to said subject.

12. The method of claim 11 where said secondary therapeutic agent is selected from the group consisting of metformin, sulforylurea and insulin.

13. The method of claim 7 where said disease state associated with insulin resistance is selected from the group consisting of: metabolic syndrome, pre-diabetes, polycystic ovary syndrome, type 2 diabetes, dyslipidemia, obesity, infertility, inflammatory disorders, cancer, inflammatory diseases, Alzheimer's disease, hypertension, atherosclerosis, cardiovascular disease and peripheral vascular disease.

14. A method for identifying a compound which activates an insulin responsive gene, said method comprising:
a. providing a cell expressing at least a portion of said insulin responsive gene, said insulin responsive gene selected from the group consisting of: MINOR, TR3 and MINOR and TR3;
b. incubating said cell with said compound and;
c. measuring a response to said compound.

15. The method of claim 14 where said response is an increase in the expression of at least one of MINOR or TR3.

16. The method of claim 14 where said response is selected from the group consisting of: an increase in the expression of a polypeptide encoded by said insulin responsive gene, an increase in the activity of a polypeptide encoded by said insulin responsive gene, a functional response mediated by said insulin responsive gene.

17. The method of claim 16 where said functional response is at least one of the responses selected from the group consisting of: increased glucose uptake, increased expression of a transporter involved in glucose regulation, increased translocation of a transporter involved in glucose regulation to the cell membrane and increased activity of downstream signal transduction pathways regulated by said insulin responsive gene.

18. The method of claim 17 where said transporter is a GLUT4 transporter.

19. A method for identifying a compound useful for the treatment or prevention of insulin resistance, said method comprising the step of determining whether the compound interacts with the polypeptide product of a gene selected from the group consisting of: MINOR, TR3 and MINOR and TR3.

20. The method of claim 19 where said interaction is a direct interaction or an indirect interaction.

21. A method for treating or preventing insulin resistance, said method comprising administering a therapeutically effective amount of a compound which was identified by the method of claim 19 to a subject in need of said treatment or prevention.

22. A method for identifying a compound useful for the treatment or prevention of insulin resistance, said method comprising the step of determining whether the compound activates a gene selected from the group consisting of: MINOR, TR3 and MINOR and TR3.

23. A method for treating or preventing insulin resistance, said method comprising administering a therapeutically effective amount of a compound which was identified by the method of claim 22 to a subject in need of said treatment or prevention.

24. A method for identifying a compound useful for the treatment or prevention of a disease state associated with insulin resistance, said method comprising the step of determining whether the compound interacts with the polypeptide product of a gene selected from the group consisting of: MINOR, TR3 and MINOR and TR3.

25. The method of claim 24 where said interaction is a direct interaction or an indirect interaction.

26. The method of claim 24 where said disease state associated with insulin resistance is selected from the group consisting of: metabolic syndrome, pre-diabetes, polycystic ovary syndrome, type 2 diabetes, dyslipidemia, obesity, infertility, inflammatory disorders, cancer, inflammatory diseases, Alzheimer's disease, hypertension, atherosclerosis, cardiovascular disease and peripheral vascular disease.

27. A method for treating or preventing a disease state associated with insulin resistance, said method comprising administering a therapeutically effective amount of a compound which was identified by the method of claim 24 to a subject in need of said treatment or prevention.

28. The method of claim 27 where said disease state associated with insulin resistance is selected from the group consisting of: metabolic syndrome, pre-diabetes, polycystic ovary syndrome, type 2 diabetes, dyslipidemia, obesity, infertility, inflammatory disorders, cancer, inflammatory diseases, Alzheimer's disease, hypertension, atherosclerosis, cardiovascular disease and peripheral vascular disease.

29. A method for identifying a compound useful for the treatment or prevention of a disease state associated with insulin resistance, said method comprising the step of determining whether the compound activates a gene selected from the group consisting of: MINOR, TR3 and MINOR and TR3.

30. The method of claim 29 where said disease state associated with insulin resistance is selected from the group consisting of: metabolic syndrome, pre-diabetes, polycystic ovary syndrome, type 2 diabetes, dyslipidemia, obesity, infertility, inflammatory disorders, cancer, inflammatory diseases, Alzheimer's disease, hypertension, atherosclerosis, cardiovascular disease and peripheral vascular disease.

31. A method for treating or preventing a disease state associated with insulin resistance, said method comprising administering a therapeutically effective amount of a compound which was identified by the method of claim 29 to a subject in need of said treatment or prevention.

32. The method of claim 31 where said disease state associated with insulin resistance is selected from the group consisting of: metabolic syndrome, pre-diabetes, polycystic ovary syndrome, type 2 diabetes, dyslipidemia, obesity, infertility, inflammatory disorders, cancer, inflammatory diseases, Alzheimer's disease, hypertension, atherosclerosis, cardiovascular disease and peripheral vascular disease.
